(19) United States
(12) Patent Application Publication
(10) Pub. No.: US 2011/0097285 A1
(43) Pub. Date: Apr. 28, 2011

(54) Multiplex dentifrice compositions

(75) Inventor: Henryk Malak, Ellicott City, MD (US)

(73) Assignee: American Environmental Systems, Inc.

(21) Appl. No.: 12/592,193

(22) Filed: Nov. 23, 2009

Related U.S. Application Data
(60) Provisional application No. 61/279,902, filed on Oct. 28, 2009.

(51) Int. Cl. A61K 8/22; A61Q 11/00

(52) U.S. Cl. 424/53

(57) ABSTRACT

This invention provides a novel aqueous dentifrice composition having multiplex effects on desulfurization of teeth, oral tissue, and on other dental and oral/body care and treatments. The dentifrice composition combined with electromagnetic energy and/or with treatment substances provides advanced means for daily oral/body care and treatments.

### CROSS REFERENCE TO RELATED APPLICATIONS

This application claims the benefit of U.S. Provisional Application Ser. No. 61/279,902 filed Oct. 28, 2009, entitled “Dentifrice liquid/gel/paste composition”.

### STATEMENT REGARDING FEDERALLY SPONSORED RESEARCH

There is NO claim for federal support in research or development of this product.

### FIELD OF THE INVENTION

The herein disclosed invention finds applicability of chemistry in the field of daily hygiene and medical care, and quality of human health.

### BACKGROUND OF THE INVENTION

Most dental and oral care problems are related to tooth remineralization and demineralization, and oral bacteria. Remineralization and demineralization have a significant impact on the strength and hardness of dental enamel. Remineralization and demineralization are processes that occur on the tooth surface. Since there is no connection between the bloodstream and tooth enamel, eating mineral supplements have no impact on the remineralization process. Demineralization is the process of removing mineral ions from dental enamel, essentially “dissolving the enamel.” Dental enamel is a crystalline latticework made up of various minerals, primarily a complex calcium phosphate mineral called hydroxyapatite. A substantial number of mineral ions can be lost from the hydroxyapatite latticework without destroying its structural integrity. Although the integrity of the tooth is still intact, a patient may experience hot, cold, pressure or pain much more readily than before demineralization. A severely demineralized spot on a tooth that hasn't yet formed a cavity may have the symptoms of a cavity. In some cases, when too many minerals are dissolved from an area of the hydroxyapatite's latticework, a cavity results. Fortunately, the latticework can be strengthened and restored through the natural process of remineralization by eating unprocessed foods rich in minerals, such as natural and organic foods. Remineralization is the process of restoring mineral ions to the tooth structure, and can be compared to replacing the missing links in a chain. The lost mineral ions must be replaced with ions of the same shape, size and electrical charge. Remineralization involves carbon dioxide from breath and water from saliva to create a mild, unstable carbonic acid that is at the core of the natural remineralization process. Minerals in saliva present from food are dissolved by the carbonic acids. In addition, carbonic acid quickly and easily converts to carbon dioxide and water. When this happens, the dissolved mineral ions precipitate out as solid mineral ions again, but not always as the original mineral molecules. If a particular mineral ion is near a demineralized portion of the hydroxyapatite crystal that requires that ion, the ion is incorporated into the dental enamel. Though natural remineralization is always taking place, the level of activity varies according to mineral and bacterial concentrations in saliva and in the mouth, respectively.

Tooth demineralization is caused by specific types of acid-producing bacteria (mutans streptococci and lactobacilli) that cause damage in the presence of fermentable carbohydrates such as sucrose, fructose, and glucose. The mineral content of teeth is sensitive to increases in acidity from the production of lactic acid. Specifically, a tooth (which is primarily mineral in content) is in a constant state of back-and-forth demineralization and remineralization between the tooth and surrounding saliva. When the pH at the surface of the tooth drops below 5.5, demineralization proceeds faster than remineralization (i.e. there is a net loss of mineral structure on the tooth's surface). This results in the ensuing decay. Depending on the extent of tooth destruction, various treatments can be used to restore teeth to proper form, function, and aesthetics, but there is no known methods and products to regenerate large amounts of tooth structure. Instead, dental health organizations advocate preventive and prophylactic measures, such as regular oral hygiene and dietary modifications, to avoid dental caries.

Predominantly, the tooth demineralization, dental caries, bad mouth smells and other dental and oral tissue problems are related to volatile sulfur compounds that are waste products created by anaerobic oral bacteria as they digest proteins. One of the best strategies to reduce these problems would be to remove volatile sulfur compounds from teeth and oral tissue. Such strategy fulfills a need for a new dentifrice that is disclosed in the present invention.

### SUMMARY OF THE INVENTION

The invention discloses a novel aqueous dentifrice composition of a peroxide compound and a salt, which has multiplex effects on desulfurization of teeth and oral tissue, deacidification of teeth and oral tissue, tooth whitening, tooth remineralization, tooth desensitization, and dental and oral hygiene and treatment. The concentrations of the peroxide compound and salt in the composition are within 0.01% to 50% and 0.001% to 40%, respectively. These multiplex effects would not undergo if each component of the dentifrice composition would be used separately. The dentifrice composition is very active at body temperature and it is not active at room or lower temperatures, which makes the dentifrice composition ideal as a dental or oral product.

Furthermore, the disclosed dentifrice composition is supported by electromagnetic energy that enhances multiple effects of the dentifrice composition on desulfurization and deacidification of teeth and oral tissue, and on other dental and oral care matters.

### DETAIL DESCRIPTION OF THE INVENTION

In a present invention is proposed a novel aqueous dentifrice composition having multiplex effects on desulfurization of teeth and oral tissue. The dentifrice composition comprises of a peroxide compound and a salt at concentrations in the composition within 0.01% to 50% and 0.001% to 40%, respectively. A presence of the both peroxide compound and salt in the composition induces multiplex effects on desulfurization processes of teeth and oral tissue, the effects that would not undergo if each component of the composition, the peroxide compound and a salt, would be used separately. The multiplex effects of the disclosed dentifrice composition on desulfurization (hydrodesulfurization) are related to reducing sulfur content in tooth plaque/bacteria, teeth and oral tissue, which is resulting in reducing odor and deacidification of teeth and oral tissue. These multiplex effects are very pronounced at body temperatures and they are very silent at room and lower temperatures. Therefore, the peroxide compound and salt can be mixed together even few weeks before the use, if the dentifrice composition would be stored at room or lower temperatures.

The variation of concentrations of the peroxide compound and salt in dentifrice composition are related to a type of applications. If dental or oral care requires preventive treatments, the dentifrice composition can be used with lower concentrations of the peroxide compound and salt. But, if dental or oral care requires invasive treatments, the dentifrice composition has to be used with higher concentrations of the peroxide compound and salt.

One of the preferable embodiments in the invention is the use a sea salt in the dentifrice composition, due to vital microelements in the sea salt that are essential in tooth remineralization. But the use of other salts, like an unrefined salt, refined salt or enriched salt are part of invention too.

Another preferable embodiment in the invention is the use a hydrogen peroxide as the peroxide compound in the dentifrice composition; however any of the peroxide compounds that are containing an oxygen-oxygen single bond are part of invention. The dentifrice composition may comprise a single peroxide compound or a multiple peroxide compound.

Another preferable embodiment in the invention is the use of an electromagnetic energy along with the dentifrice composition for desulfurization of teeth and oral tissue, and for other dental and oral care treatments. The dentifrice composition in the presence of electromagnetic energy is becoming more reactive that leads to the enhanced multiplex effects, which would allow of using the dentifrice composition with lower concentrations of the peroxide and salt. In the invention is proposed to use the electromagnetic energy at wavelengths within 0.1 nm to 20,000 nm. The electromagnetic energy at lower wavelengths or higher wavelengths enhances peroxide compound chemical reactivity in the dentifrice composition by quantum effects or thermal effects, respectively.

Another preferable embodiment in the invention is the use of a treatment substance along with the dentifrice composition for desulfurization of teeth and oral tissue, and for other dental and oral care treatments. The use of the treatment substance in the presence of the multiplex effects of the dentifrice composition may provide more effective treatment to dental and oral care matters.

The disclosed dentifrice composition is ready to be used for dental and oral tissue care. When the dentifrice composition is applied to teeth or oral tissue, it creates self-sustainable thick foam that is allowing for effective teeth and gums cleaning and treatment. At higher concentrations of salt in the dentifrice composition, the salt is forming nano-micrometers size crystals that can be used in the dentifrice composition as an abrasive means. However, the invention did not exclude the use additionally another abrasive substance or gel type substance in the dentifrice composition.

The multiplex effects of the dentifrice composition is additionally claimed in the invention for the use in whitening, cleaning and hygiene teeth and tissue, tooth remineralization, tooth desensitization, and gum/tissue treatment. The tooth whitening by the dentifrice composition is not only related to whitening effect of the peroxide but also to tooth remineralization by the salt, and the tooth remineralization also leads to the tooth desensitization.

What is claimed is:

1. An aqueous dentifrice composition having multiplex effects on desulfurization of teeth and oral tissue comprising of: a peroxide compound and a salt, wherein the peroxide compound concentration in the composition is about 0.01% to about 50% by weight and the salt concentration in the composition is about 0.001% to about 40% by weight.

2. The dentifrice composition of claim 1, wherein the salt is an unrefined salt, refined salt, sea salt, or enriched salt.

3. The dentifrice composition of claim 1, wherein the peroxide compound is a single peroxide compound or a multiple peroxide compound.

4. The dentifrice composition of claim 1, wherein the at least peroxide compound is hydrogen peroxide.

5. The dentifrice composition of claim 1, wherein the dentifrice composition further comprising an electromagnetic energy.

6. The dentifrice composition of claim 5, wherein the electromagnetic energy is at wavelength or wavelengths within 0.1 nm to 20,000 nm.

7. The dentifrice composition of claim 1, wherein the dentifrice composition further comprising a treatment substance.

8. The dentifrice composition of claim 1, wherein the dentifrice composition further comprising a gel substance.

9. The dentifrice composition of claim 1, wherein the dentifrice composition has further multiplex effects on tooth whitening, tooth and oral tissue deacidification, cleaning and hygiene, tooth remineralization, tooth desensitization, or gum/tissue treatment.
